(19) United States
(12) Patent Application Publication
(10) Pub. No.: US 2015/0073452 A1
(43) Pub. Date: Mar. 12, 2015

(54) LIQUID EJECTING APPARATUS, LIQUID EJECTING METHOD, AND MEDICAL INSTRUMENT

(71) Applicant: SEIKO EPSON CORPORATION, Tokyo (JP)

(72) Inventors: Kazuaki UCHIDA, Fujimi-machi (JP); Hirokazu SEKINO, Chino-shi (JP)

(21) Appl. No.: 14/483,338

(22) Filed: Sep. 11, 2014

(30) Foreign Application Priority Data
Sep. 11, 2013 (JP) 2013-188270

(51) Int. Cl. A61B 17/3203; B05B 12/06; A61B 19/00

(52) U.S. Cl. CPC A61B 17/3203; A61B 19/56; B05B 12/06; A61B 2017/00402
USPC 606/167; 239/101

(57) ABSTRACT

A liquid is supplied to a liquid chamber, the pressure in a liquid chamber fluctuates according to a drive signal, and the liquid of the liquid chamber is ejected from an ejection port at the tip of an ejection pipe by fluctuation of the pressure in the liquid chamber. At this time, the relationship between the amount of variation of the moving speed of the ejection port and the amount of change of a parameter (drive signal) involved in fluctuation of the state of the liquid ejected from the ejection port is set, and the parameter is changed according to the relative speed of the ejection port using the set relationship.

This application claims the benefit of Japanese Patent Application No. 2013-188270, filed on Sep. 11, 2013. The content of aforementioned application is incorporated by reference herein in its entirety.

### BACKGROUND

1. Technical Field

The present invention relates to ejection of a liquid.

2. Related Art

In a liquid ejecting apparatus which is used as a medical instrument, a technique for controlling energy of a liquid ejected from an ejection port is known (for example, JP-A-2010-51896). The ejection of the liquid from the ejection port includes a continuous type which continuously performs ejection and an intermittent type which intermittently performs ejection. In all cases, parameters, such as an ejection speed and a flow rate, are controlled, thereby adjusting capacity, such as resection capacity of the medical instrument.

In recent years, in the liquid ejecting apparatus which is used as the medical instrument, a method which measures an acceleration of the ejection port and selects a mode of liquid ejection based on the acceleration has been suggested (for example, JP-A-2012-143374).

This liquid ejecting apparatus is excellent in that a predetermined action can be affected to a target using a medium, such as a liquid, and widespread use is possible. In a liquid ejecting apparatus of a type which is switched to a specific ejection mode according to the moving speed of the ejection port, high safety of the medical instrument is secured. The inventors have studied the aspects of use of the apparatus and have found an easy-to-use configuration. In addition, reduction in size of the apparatus, low cost, resources saving, ease of manufacturing, improvement of usability, and the like are required. The inventors have attempted to solve these problems.

### SUMMARY

An advantage of some aspects of the invention is to solve at least a part of the problems described above, and the invention can be implemented as the following forms or application examples.

### Application Example 1

A first aspect of the invention provides a liquid ejecting apparatus which ejects a liquid. The liquid ejecting apparatus includes a liquid ejecting mechanism which receives a drive signal as input and makes the pressure of the liquid in an internal liquid chamber fluctuate according to the drive signal to make the liquid be ejected from an ejection port. The liquid ejecting apparatus may be configured such that the drive signal is changed according to the moving speed of the liquid ejecting mechanism and the relationship between the amount of variation of the moving speed and the amount of change of the drive signal is able to be set.

According to the liquid ejecting apparatus, it is possible to set the relationship between the amount of variation of the moving speed and the amount of change of the drive signal. In the liquid ejecting apparatus, the drive signal is changed according to the moving speed of the liquid ejecting mechanism using the set relationship. As a result, it is possible to easily perform the ejection of the liquid with a predetermined relationship.

### Application Example 2

The liquid ejecting apparatus according to the aspect of the invention described above may further include a user interface which receives an instruction of a set value, and a setting unit which sets the relationship between the amount of variation of the moving speed and the amount of change of the drive signal according to the set value instructed by the user interface.

According to the liquid ejecting apparatus, it is possible to set the relationship between the amount of variation of the moving speed and the amount of change of the drive signal by the set value instructed through the user interface, and to allow the user to easily establish a desired relationship.

### Application Example 3

The liquid ejecting apparatus according to the aspect of the invention described above may further include a storage unit which stores a plurality of relationships between the amount of variation of the moving speed and the amount of change of the drive signal, and a setting unit which sets the relationship between the amount of variation of the moving speed and the amount of change of the drive signal to one relationship selected from the storage unit.

According to the liquid ejecting apparatus, since a plurality of relationships are stored in advance and one relationship selected among the plurality of relationships is set, it is possible to easily establish a desired relationship.

### Application Example 4

In the liquid ejecting apparatus according to the aspect of the invention described above, the drive signal may include at least one of the frequency of the drive signal and the voltage of the drive signal.

In the liquid ejecting apparatus, since an easy-to-control element, such as the frequency of the drive signal or the voltage of the drive signal, is used, it is possible to easily control the ejection state of the liquid. The drive signal is changed to change the state of the liquid ejected from the ejection port, and if the relationship with the amount of change of a parameter capable of establishing the change can be set, a parameter other than the drive frequency of the drive signal or the voltage of the drive signal may be used. For example, the amount of supply of the liquid to the liquid chamber, the representative volume of the liquid chamber, or the like may be used to change the ejection state of the liquid from the ejection port.

### Application Example 5

In the liquid ejecting apparatus according to the aspect of the invention described above, when the moving speed is a first speed, the drive signal may be determined to be a first value, and when the moving speed is a second speed faster than the first speed, the drive signal may be determined to be a second value at which power by the ejected liquid is higher than power of the first value.

According to the liquid ejecting apparatus, since power increases with an increase in the moving speed, a difference in power per unit distance (or unit time) is suppressed.

### Application Example 6

In the liquid ejecting apparatus according to the aspect of the invention described above, the setting unit may set the relationship between the increase of the second speed with respect to the first speed of the moving speed and the increase of the second value with respect to the first value of the drive signal.

According to the liquid ejecting apparatus, since the relationship between the increase of the moving speed and the increase of the drive signal is set, the relationship between both the increase of the moving speed and the increase of the drive signal can be easily understood.

### Application Example 7

In the liquid ejecting apparatus according to the aspect of the invention described above, the setting unit may set one of a first mode, in which the increase ratio of the increase of the value and the increase of the moving speed is set to be less than 1, a second mode, in which the increase ratio is set to 1, and a third mode, in which the increase ratio is set to be greater than 1, according to information input by the user interface.

According to the liquid ejecting apparatus, since one of the three modes can be easily selected by the user interface, it is possible to easily select a desired relationship.

### Application Example 8

The liquid ejecting apparatus according to the aspect of the invention described above may further include a liquid supply unit which supplies the liquid to the liquid chamber, and the supply flow rate of the liquid to the liquid chamber by the liquid supply unit may be adjusted according to change of the drive signal.

In the liquid ejecting apparatus, if the drive signal is changed, the amount of the liquid to be supplied to the liquid chamber may vary. For this reason, the supply flow rate of the liquid to the liquid chamber is adjusted according to change of the drive signal, making it possible to supply neither too much nor too little of the liquid.

### Application Example 9

Another aspect of the invention provides a medical instrument. The medical instrument may include a liquid supply unit which supplies the liquid to the liquid chamber, and the liquid supply unit may supply a liquid for medical use to the liquid chamber. The medical instrument can appropriately set the relationship between the moving speed of the ejection port of the liquid ejecting apparatus and the drive signal and can use the relationship in medicine.

### Application Example 10

In the medical instrument according to the aspect of the invention described above, the liquid ejecting apparatus may apply pulsation to the liquid to perform the ejection of the liquid. When pulsation is applied to the liquid, it becomes possible to perform incision or resection more appropriately.

### Application Example 11

Still another aspect of the invention provides a liquid ejecting apparatus including a liquid ejecting mechanism and a change unit. The liquid ejecting mechanism may receive a drive signal as input and may make the pressure of a liquid in an internal liquid chamber fluctuate according to the drive signal to make the liquid be ejected from an ejection port. The change unit may change a parameter involved in fluctuation of the state of the liquid ejected from the ejection port according to the moving speed of the liquid ejecting mechanism. In the liquid ejecting apparatus, the relationship between the moving speed and the parameter corresponding to the moving speed is able to be set.

According to the liquid ejecting apparatus, it is possible to set the relationship between the amount of variation of the moving speed and the amount of change of the parameter. In the liquid ejecting apparatus, the parameter is changed according to the moving speed of the liquid ejecting mechanism using the set relationship. As a result, it is possible to easily perform the ejection of the liquid with a relationship set in advance.

### Application Example 12

Yet another aspect of the invention provides a liquid ejecting method. The liquid ejecting method makes a liquid be supplied to a liquid chamber, makes a pressure in the liquid chamber fluctuate according to a drive signal, and makes the liquid of the liquid chamber be ejected from an ejection port at the tip of an ejection pipe by fluctuation of the pressure in the liquid chamber. The liquid ejecting method may include setting the relationship between the amount of variation of the moving speed of the ejection port and the amount of change of a parameter involved in fluctuation of the state of the liquid ejected from the ejection port, and changing the parameter according to the moving speed using the set relationship.

According to the liquid ejecting method, the relationship between the amount of variation of the moving speed and the amount of change of the parameter involved in fluctuation of the state of the liquid can be set by the setting unit. The liquid ejecting apparatus uses the set relationship to change the state of the liquid ejected from the ejection port according to the moving speed of the ejection port. As a result, it is possible to easily perform the ejection of the liquid with a relationship set in advance.

### Other Application Examples

The liquid ejecting apparatus according to the aspect of the invention described above may further include a housing which accommodates hardware configured to output a signal to the liquid ejecting mechanism, and an operating unit which is provided in the housing and instructs to change the relationship. In the liquid ejecting apparatus, since the operating unit and the housing which accommodates hardware configured to output a signal to the liquid ejecting mechanism can be united as a single body, it is possible to achieve ease of handling of the apparatus.

Alternatively, the liquid ejecting apparatus according to the aspect of the invention described above may further include a pedal which receives an instruction to eject the liquid and outputs the instruction to the liquid ejecting mechanism, and an operating unit which is provided near the pedal and selects the setting of the relationship. In the liquid ejecting apparatus, since it is possible to perform an instruction of ejection by the pedal and to perform the selection of the setting near the pedal, convenience is excellent.

A plurality of components provided to each of the aspects of the invention described above are not necessarily essential, and in order to solve all or a part of the problems described above or in order to achieve all or a part of the advantages described in the specification, it is possible to arbitrarily perform modification, elimination, replacement with another new component, and partial deletion of restriction content on some of the plurality of components. Furthermore, in order to solve all or a part of the problems described above or in order to achieve all or apart of the advantages described in the specification, it is also possible to combine some or all of the technical features included in one of the aspects of the invention with some or all of the technical features included in another aspect of the invention to thereby form an independent aspect of the invention.

The invention can be implemented in various forms other than the apparatus. The invention can be implemented in the forms of, for example, a manufacturing method of a liquid ejecting apparatus, a control method of a liquid ejecting apparatus, a computer program for implementing the control method, and a non-temporary recording medium on which the computer program is recorded.

### BRIEF DESCRIPTION OF THE DRAWINGS

The invention will be described with reference to the accompanying drawings, wherein like numbers reference like elements.

FIG. 1 is a schematic configuration diagram of a liquid ejecting apparatus (medical instrument).

FIG. 2 is an internal configuration diagram of a liquid ejecting mechanism.

FIG. 3 is an explanatory view illustrating the appearance of a control unit.

FIG. 4 is a block diagram showing the internal configuration of a control unit.

FIG. 5 is a graph showing a drive waveform.

FIG. 6 is a flowchart (first embodiment) showing ejection processing.

FIG. 7 is a graph showing the relationship between an ejection port speed S and a drive frequency F with a set value a as a parameter.

FIG. 8 is a flowchart showing a 20 msec interrupt routine for setting a set value a.

FIG. 9 is a flowchart showing a memory switch interrupt routine which changes a set value of a preset switch.

FIG. 10 is a perspective view showing the appearance of a foot switch in a second embodiment.

FIG. 11 is an explanatory view illustrating the relationship between a speed S in a right-left (up-down) direction and a drive frequency F.

FIG. 12 is an explanatory view illustrating another relationship between a speed S and a drive frequency F (or peak voltage E).

### DESCRIPTION OF EXEMPLARY EMBODIMENTS

### A. First Embodiment

### A1. Overall Configuration

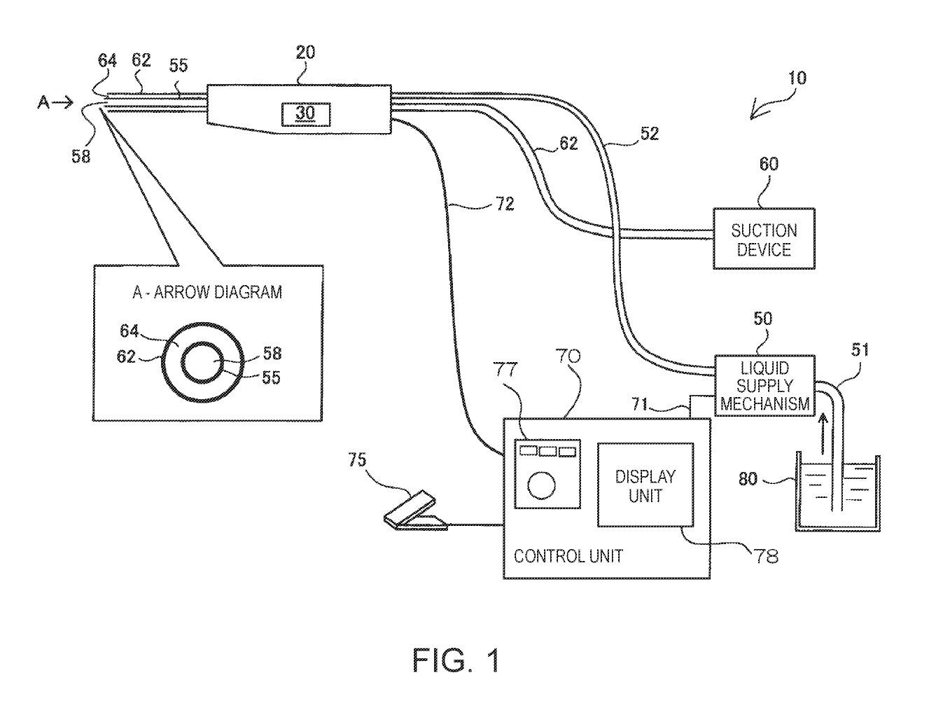

A first embodiment will be described. FIG. 1 shows the configuration of a liquid ejecting apparatus 10. The liquid ejecting apparatus 10 is a medical instrument which is used in a medical institution and has a function of ejecting a liquid to an affected part to incise or resect the affected part.

The liquid ejecting apparatus 10 has a liquid ejecting mechanism (handpiece) 20, a liquid supply mechanism 50, a suction device 60, a control unit 70, and a liquid container 80. The liquid supply mechanism 50 and the liquid container 80 are connected together by a connection tube 51. The liquid supply mechanism 50 and the liquid ejecting mechanism 20 are connected together by a liquid supply flow channel 52. The connection tube 51 and the liquid supply flow channel 52 are formed of resin. The connection tube 51 and the liquid supply flow channel 52 may be formed of a material (for example, metal) other than resin.

The liquid container 80 stores physiological saline. Instead of physiological saline, pure water or a chemical may be used. The liquid supply mechanism 50 supplies the liquid sucked from the liquid container 80 through the connection tube 51 to the liquid ejecting mechanism 20 through the liquid supply flow channel 52 by driving of an internal pump.

The liquid ejecting mechanism 20 is a tool which is operated in a hand of a user of the liquid ejecting apparatus 10. The liquid ejecting mechanism 20 can intermittently eject the liquid by an internal pulsation generation unit 30. The user hits an affected part with the liquid intermittently ejected, thereby incising or resecting the affected part. The details of a pulsation generation mechanism and control for ejecting the liquid from the liquid ejecting mechanism 20 will be described below.

The control unit 70 includes an operating unit 77 and a display unit 78. The control unit 70 is connected to the liquid supply mechanism 50 through a control cable 71, is connected to the liquid ejecting mechanism 20 through a signal cable 72, and is further connected to a foot switch 75. The control unit 70 can control the liquid supply mechanism 50 through the control cable 71 and controls the flow rate of the liquid supplied to the pulsation generation unit 30. The control unit 70 can transmit a drive signal to the pulsation generation unit 30 embedded in the liquid ejecting mechanism 20 through the signal cable 72. If the user turns on the foot switch 75, the control unit 70 performs control such that the liquid supply mechanism 50 executes the supply of the liquid to the pulsation generation unit 30, and transmits the drive signal to the pulsation generation unit 30 to generate pulsation in the pressure of the liquid supplied to the pulsation generation unit 30. The internal configuration of the control unit 70 or processing using the operating unit 77 or the like will be described in detail.

The suction device 60 is provided to suck the liquid around an ejection port 58 or a resected substance. The suction device 60 and the liquid ejecting mechanism 20 are connected together by a suction flow channel 62. The suction device 60 constantly sucks the inside of the suction flow channel 62 while the switch for operating the suction device 60 is turned on. The suction flow channel 62 passes through the liquid ejecting mechanism 20 and is opened near the tip of an ejection pipe 55.

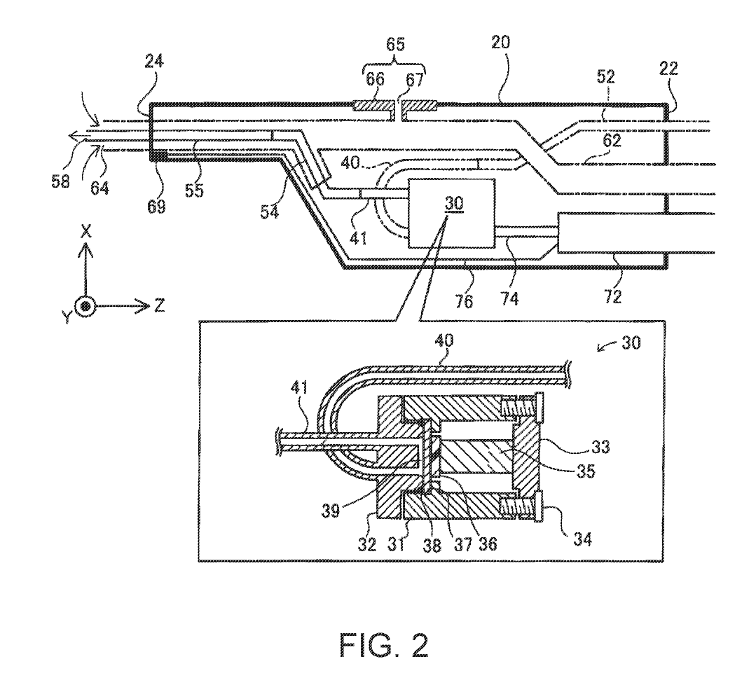

The suction flow channel 62 covers the ejection pipe 55 extending from the tip of the liquid ejecting mechanism 20. For this reason, as shown in an A-arrow diagram of FIG. 1, the wall of the ejection pipe 55 and the wall of the suction flow channel 62 form a substantially concentric cylinder. A flow channel through which a sucked substance sucked from a suction port 64 at the tip of the suction flow channel 62 is formed between the outer wall of the ejection pipe 55 and the inner wall of the suction flow channel 62. The sucked substance is sucked to the suction device 60 through the suction flow channel 62. The suction is adjusted by a suction adjustment mechanism 65 described below referring to FIG. 2.

### A2. Internal Configuration of Liquid Ejecting Mechanism

FIG. 2 shows the internal structure of the liquid ejecting mechanism 20. The liquid ejecting mechanism 20 includes a pulsation generation unit 30, an entrance flow channel 40, an exit flow channel 41, a connection tube 54, an acceleration sensor 69, and a suction force adjustment mechanism 65.

The pulsation generation unit 30 generates pulsation in the pressure of the liquid supplied from the liquid supply mechanism 50 to the liquid ejecting mechanism 20 through the liquid supply flow channel 52. The liquid in which pulsation of the pressure is generated is supplied to the ejection pipe 55. The liquid supplied to the ejection pipe 55 is intermittently ejected from the ejection port 58. The ejection pipe 55 is formed of stainless steel. The ejection pipe 55 may be formed of other materials having predetermined rigidity or more, for example, other metals, such as brass, or reinforced plastics.

As shown in an enlarged view on the lower side of FIG. 2, the pulsation generation unit 30 includes a first case 31, a second case 32, a third case 33, bolts 34, a piezoelectric element 35, a reinforcing plate 36, a diaphragm 37, a packing 38, an entrance flow channel 40, and an exit flow channel 41. The first case 31 is a tubular member. The first case 31 is closed as a whole in a state where the second case 32 is bonded to one end portion of the first case 31 and the third case 33 is fixed to the other end portion by the bolts 34. The piezoelectric element 35 is disposed in a space formed inside the first case 31.

The piezoelectric element 35 is a laminated piezoelectric element. One end of the piezoelectric element 35 is fixed to the diaphragm 37 through the reinforcing plate 36. The other end of the piezoelectric element 35 is fixed to the third case 33. The diaphragm 37 is produced by a metal thin film. The peripheral portion of the diaphragm 37 is fixed to the first case 31 and is sandwiched between the first case 31 and the second case 32. A liquid chamber 39 is formed between the diaphragm 37 and the second case 32.

The piezoelectric element 35 receives the drive signal from the control unit 70 through the signal cable 72 as input. The signal cable 72 is inserted from a rear end portion 22 of the liquid ejecting mechanism 20. The signal cable 72 accommodates two electrode lines 74 and one signal line 76 for an acceleration sensor. The electrode lines 74 are connected to the piezoelectric element 35 in the pulsation generation unit 30. The piezoelectric element 35 expands and contracts based on the drive signal transmitted from the control unit 70. The volume of the liquid chamber 39 fluctuates by the expansion and contraction of the piezoelectric element 35.

The entrance flow channel 40 into which the liquid flows is connected to the second case 32. The entrance flow channel 40 is bent in a U shape and extends toward the rear end portion 22 of the liquid ejecting mechanism 20. The liquid supply flow channel 52 is connected to the entrance flow channel 40. The liquid supplied from the liquid supply mechanism 50 is supplied to the liquid chamber 39 through the liquid supply flow channel 52.

If the piezoelectric element 35 expands and contracts at a predetermined drive frequency, the diaphragm 37 vibrates. If the diaphragm 37 vibrates, the volume of the liquid chamber 39 fluctuates and the pressure of the liquid in the liquid chamber is pulsed. The pressurized liquid flows out of the exit flow channel 41 connected to the liquid chamber 39.

The ejection pipe 55 is connected to the exit flow channel 41 through the metallic connection tube 54. The liquid flowing out of the exit flow channel 41 is ejected from the ejection port 58 through the connection tube 54 and the ejection pipe 55.

The suction force adjustment mechanism 65 adjusts a force when the suction flow channel 62 sucks the liquid or the like from the suction port 64. The suction force adjustment mechanism 65 includes an operating unit 66 and a hole 67. The hole 67 is a through hole which connects the suction flow channel 62 and the operating unit 66. If the user opens and closes the hole 67 with the finger of the hand holding the liquid ejecting mechanism 20, the amount of air flowing into the suction flow channel 62 through the hole 67 is adjusted by the degree of opening and closing, and accordingly, the suction force of the suction port 64 is adjusted. The adjustment of the suction force may be implemented by control of the suction device 60.

The liquid ejecting mechanism 20 includes an acceleration sensor 69. The acceleration sensor 69 is a piezoresistive three-axis acceleration sensor. The three axes are the respective axes of XYZ shown in FIG. 2. The X axis is parallel to the through direction of the hole 67, and an upward direction is a positive direction. The Z axis is parallel to the major axis direction of the ejection pipe 55, and a direction in which the liquid is ejected is a negative direction. The Y axis is defined by a right handed system based on the X axis and the Z axis.

As shown in FIG. 2, the acceleration sensor 69 is disposed near a tip portion 24 of the liquid ejecting mechanism 20. A measurement result is input to the control unit 70 through the signal line 76 for an acceleration sensor. Accordingly, the control unit 70 analyzes a signal from the acceleration sensor 69, thereby detecting the moving direction and speed of the liquid ejecting mechanism 20 in the right-left direction (y-axis direction) or the moving direction and speed of the liquid ejecting mechanism 20 in the up-down direction (x-axis direction). In this embodiment, although the motion of the ejection port 58 is found by a signal from one acceleration sensor 69, if a plurality of acceleration sensors are provided, and calculation is performed using the outputs of the plurality of acceleration sensors, the motion of the ejection port 58 can be detected with higher precision.

### A3. Configuration and Action of Control Unit

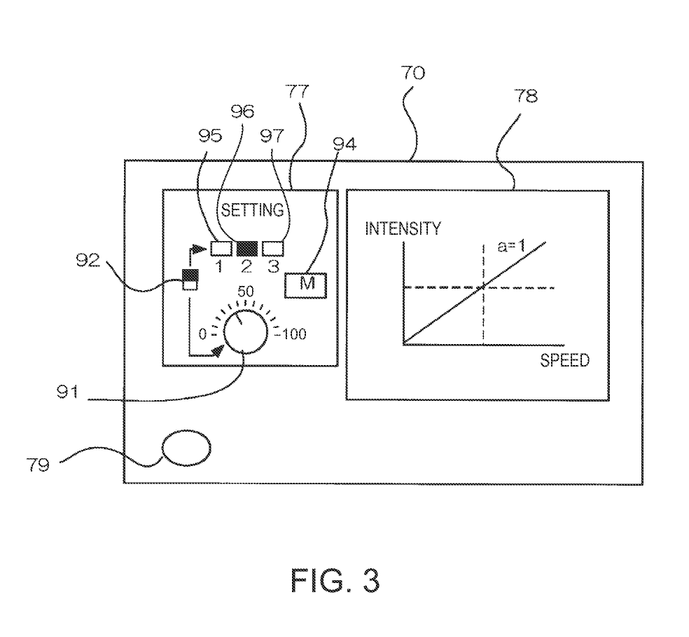

As described above, the control unit 70 performs various settings in addition to the moving speed of the ejection port 58 using the acceleration sensor 69. The appearance and the internal configuration of the control unit 70 will be described. FIG. 3 is an explanatory view showing the appearance of a full panel of the control unit 70 in this embodiment. As shown in the drawing, the control unit 70 includes a power switch 79 in addition to an operating unit 77 and a display unit 78. The operating unit 77 is provided with a rotary setter 91 which directly designates a set value a described below, a selection switch 92, three preset switches 95, 96, and 97, a memory switch 94 which causes the preset switches to store the set value, and the like. The preset switches 95 to 97 include a mechanism which is selectively turned on, and one of the switches is constantly turned on. In FIG. 3, the preset switch 96 is turned on (blackened state). The display unit 78 is a liquid crystal display panel and can display various kinds of text and images (primarily, graphs).

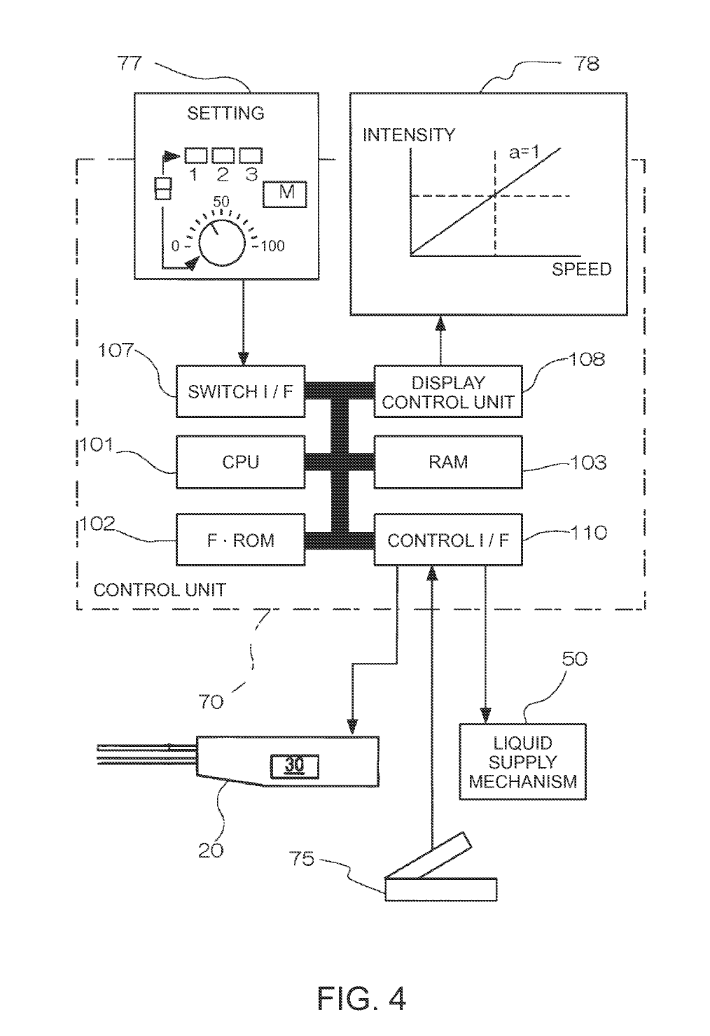

FIG. 4 shows the internal configuration of the control unit 70. The control unit 70 includes a CPU 101 which controls overall control, a flash ROM (F•ROM) 102, a RAM 103, a switch interface (switch I/F) 107, a display control unit 108, and a control interface (control I/F) 110. The F•ROM 102 is a rewriteable memory which stores a processing program of the CPU 101, a preset value of the set value a, and the like in a nonvolatile manner. The RAM 103 provides a work area when the CPU 101 executes the program. The switch I/F 107 is an interface to which a signal from the operating unit 77 is input. The display control unit 108 is a dedicated controller which is connected to the display unit 78 and controls the display of the display unit 78. The control I/F 110 is connected to the liquid ejecting mechanism 20, the liquid supply mechanism 50, and the foot switch 75, and provides an interface for exchanging signals with the respective units. These units are connected together by a bus.

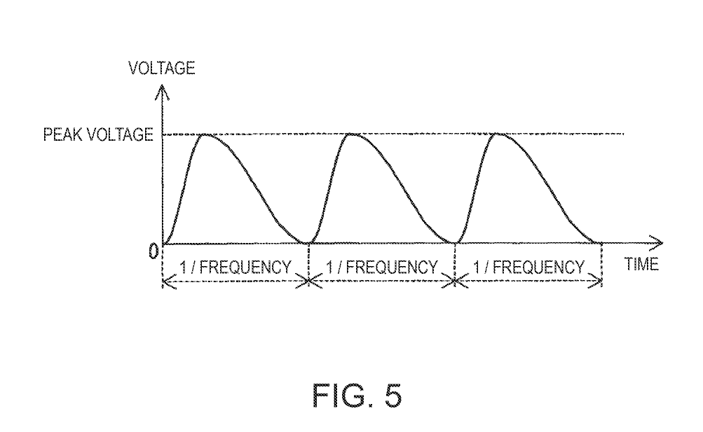

The control unit 70 controls the liquid supply mechanism 50, the pulsation generation unit 30 of the liquid ejecting mechanism 20, or the like under the control of the CPU 101, and controls the ejection of the liquid from the ejection port 58. The control for the ejection of the liquid includes the size of the liquid ejected from the ejection port 58, the intensity (energy per unit time) of the liquid, and the like. The size or ejection intensity of the liquid to be ejected is changed by adjusting the drive signal output from the control unit 70 to the piezoelectric element 35 through the electrode lines 74. FIG. 5 is a graph showing the waveform (hereinafter, referred to as “drive waveform”) of the drive signal input to the piezoelectric element 35. The vertical axis represents voltage and the horizontal axis represents time. The drive waveform is described by a combination of sinusoidal curves. The frequency (alternatively, peak voltage or the like) of the drive signal in the drive waveform varies with ejection processing described below. The liquid ejected from the ejection port 58 of the liquid ejecting mechanism 20 is pulsed according to the drive waveform. In the following description, the behavior (pulsation) of the liquid corresponding to one period of the drive waveform shown in FIG. 5 is referred to as one pulse. In one pulse, although the liquid ejected from the ejection port 58 becomes a perfect droplet and is independent, the liquid may be ejected dragging along a satellite, or the flow of the liquid from the ejection port 58 may be substantially continuous. The ejection of the liquid includes a concept of liquid discharge, liquid droplet emission, or the like.

Considering per pulse, simplistically, if the peak voltage (also referred to as intensity) of the drive signal increases, the maximum deformation of the piezoelectric element 35 increases and the contraction of the volume of the liquid chamber 39, that is, the amount of ejection per pulse increases. If the rising time of the drive signal is shortened, the deformation of the piezoelectric element 35 occurs quickly and the speed of the liquid ejected for each pulse increases. As a result, energy per pulse of the liquid to be intermittently ejected increases. Meanwhile, if the frequency (hereinafter, referred to as a drive frequency) of the drive signal increases, the number of pulses (the number of pulsations) to be ejected per unit time increases. As a result, the total amount of energy of the liquid to be ejected per unit time increases.

Next, processing of the control unit 70 will be described in detail. First, processing for controlling the intensity (energy per unit time) of the liquid to be ejected from the liquid ejecting mechanism 20 will be first described, and then, processing for setting the set value a will be described.

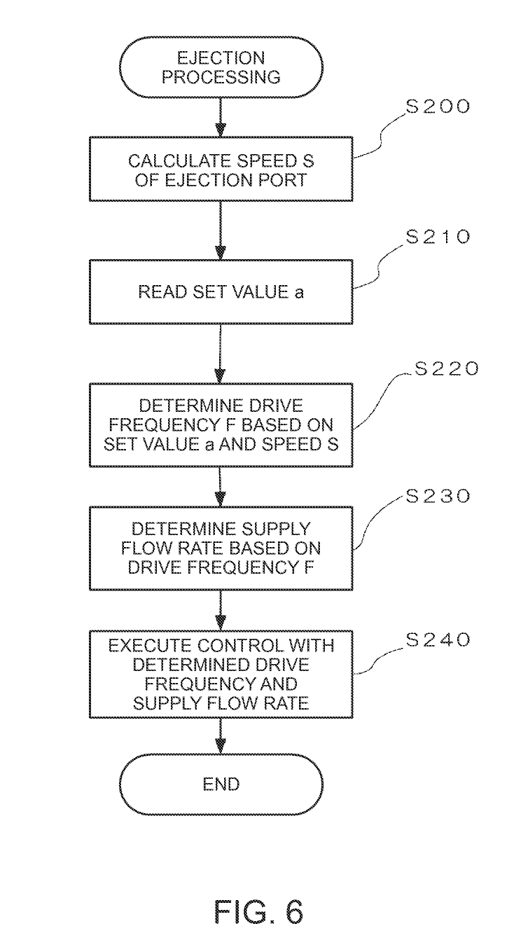

FIG. 6 is a flowchart showing ejection processing which is executed by the control unit 70. The ejection processing is repeatedly executed by the control unit 70 while the foot switch 75 is pushed down. Initially, the speed S of the ejection port 58 is calculated (Step S200). The speed S used herein is the absolute value of a speed on the XY plane. That is, the speed S is the absolute value of a speed without regard to a speed in the Z-axis direction. The speed S is calculated based on the acceleration along the three axes measured by the acceleration sensor 69.

The speed S is calculated as a parameter which affects the resection depth of the affected part. This is because a resection capacity acting on each local region of the affected part per unit time is affected by the relative speed of the ejection port 58 and the affected part. In this embodiment, on an assumption that the affected part is stationary, the speed S is handled as the moving speed of the affected part and the ejection port 58. Considering that the affected part is moved by breathing or the like, the speed S may be handled as the relative speed of the ejection port 58 and the affected part.

Subsequently, processing for reading the set value a is performed (Step S210). The set value a is a value which is set by operating the operating unit 77, and in this embodiment, is set in a range of 0.5 to 2.0. Although a method of setting the set value a will be described below in detail, here, it is assumed that the set value is set based on the states of the preset switches 95 to 97 of the operating unit 77. In a state shown in FIG. 3, that is, a state in which the preset switch 96 is pressed, the set value a is set to a value of 1.0. If the preset switch 95 is pressed, a value of 0.5 is set, and if the preset switch 97 is pressed, a value of 2.0 is set.

If the speed S and the set value a are determined, next, the drive frequency of the piezoelectric element 35 is determined based on the speed S (Step S220). The drive frequency F is determined by Expression (1).

F=a·N·S (1)

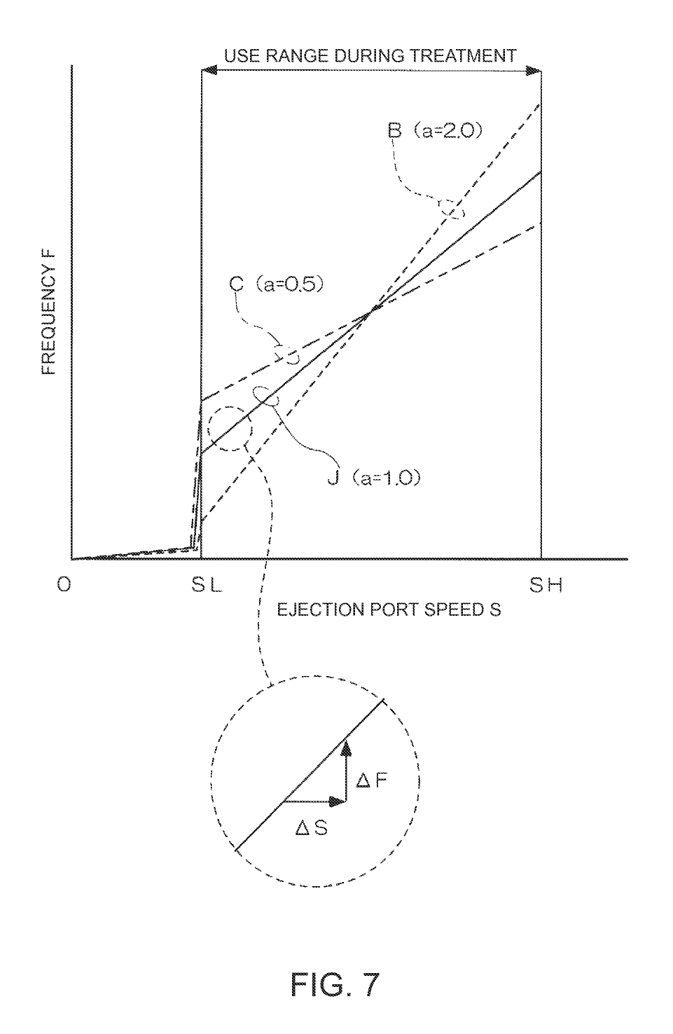

Here, a is the above-described set value, and N is a constant determined in advance. Expression (1) shows that, if the speed S increases, the drive frequency F increases in proportion to a·N. The set value a is set to one of 0.5, 1.0, and 2.0 by the states of the preset switches 95 to 97. The relationship between the speed S of the ejection port 58 and the drive frequency F is shown in FIG. 7 with the set value a as a parameter. In the drawing, a solid line J indicates the relationship when the set value a is the value of 1.0, a two-dot-chain line C indicates the relationship when the set value a is the value of 0.5, and a broken line B indicates the relationship when the set value a is the value of 2.0.

As shown in the drawing, if the speed S increases, the drive frequency F is determined to be a large value without depending on the set value a. However, if the set value a is the value of 0.5, an increase ΔF of the drive frequency F with respect to an increase ΔS of the speed S is less than when the set value a is the value of 1.0 (½), and if the set value a is the value of 2.0, the increase ΔF of the drive frequency F with respect to the increase ΔS of the speed S is greater than when the set value a is the value of 1.0 (two times). As a result, in the use range SL to SH of the speed of the ejection port 58 during treatment, when determining the drive frequency F by the speed S of the ejection port 58, the increase ratio (ΔF/ΔS) is set by the states of the preset switches 95 to 97.

Since the drive frequency F corresponds to the number of pulses of the liquid to be ejected per unit time, when the speed S increases, if the drive frequency F remains unchanged, the energy of the liquid to be applied to a unit length of a treatment part is lowered. In a sense, resection or incision capacity by the liquid ejecting mechanism 20 is degraded. In contrast, as in Expression (1), if the drive frequency F increases in proportion to the speed S, the energy of the liquid to be applied to the unit length of the treatment part increases by that much. In this embodiment, when the set value a is the value of 1.0, the energy per unit length of the treatment part is kept constant. When the set value a is the value of 0.5, the increase of the frequency necessary for making the energy per unit length of the treatment part constant is suppressed to ½ with an increase of the speed S of the ejection port 58. When the set value a is the value of 2.0, the increase of the frequency necessary for making the energy per unit length of the treatment part constant is increased to two times with an increase of the speed S of the ejection port 58.

For this reason, when the set value a is the value of 1.0, the resection or incision capacity is substantially kept constant regardless of the speed S of the ejection port 58. When the set value a is the value of 2.0, when moving the liquid ejecting mechanism 20 rapidly, the energy per unit length increases, and thus, resection or incision can be performed at higher speed. When moving the liquid ejecting mechanism 20 slowly, the energy per unit length decreases, and thus resection or incision capacity becomes insensitive and more careful treatment is possible. When the set value a is the value of 0.5, when moving the liquid ejecting mechanism 20 rapidly, the energy per unit length decreases, and thus, it is possible to avoid a possibility that resection or incision is performed to an unexpected depth with high-speed movement. When moving the liquid ejecting mechanism 20 slowly, the energy per unit length increases, and thus, resection or incision capacity increases and treatment in a wide range (to a deep place) with reliable motion is possible. These have the relationship of resection or incision capacity to motion of the liquid ejecting mechanism 20, a preferable relationship is different depending on the preference of the user, the characteristic of a use target, or the like rather than saying that any is correct. In this embodiment, this can be freely set by the states of the preset switches 95 to 97.

After the drive frequency F is determined by the speed S, next, the supply flow rate is determined based on the drive frequency F (Step S230), and control is executed such that the determined drive frequency and supply flow rate are implemented (Step S240). The supply flow rate is the volume flow rate of the liquid supplied by the liquid supply mechanism 50. If the drive frequency increases, the amount of the liquid ejected per unit time varies, and the liquid of an amount slightly exceeding a necessary flow rate is supplied from the liquid supply mechanism 50 to the liquid ejecting mechanism 20 conforming to this.

In the above-described embodiment, the increase of the drive frequency F to the speed S is set to one of the three states by the value of the set value a set by the states of the preset switches 95 to 97. A method of setting the set value a will be described below.

### A4. Setting of Set Value a

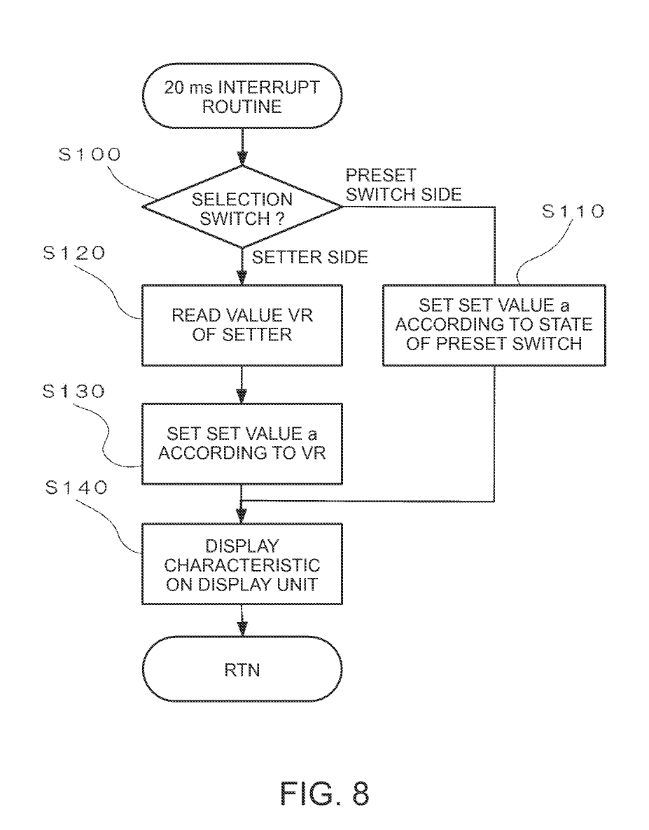

FIG. 8 is a flowchart showing an interrupt processing routine which is executed by the control unit 70 for every 20 msec. This processing is executed for every 20 msec using a timer embedded in the CPU 101 after the power switch 79 of the control unit 70 is turned on and a program of initial setting or initial inspection is executed. If the processing shown in FIG. 8 starts, first, determination is performed about the side to which the selection switch 92 of the operating unit 77 is switched (Step S100). If it is determined that the selection switch 92 is switched to the preset switch side, the CPU 101 performs processing for setting the set value a according to the states of the preset switches 95 to 97 (Step S110). Specifically, determination is performed about which of the three preset switches 95 to 97 is pressed, and if the preset switch 95 is pressed, the value set in the switch in advance is set to the set value a. Since the value of 0.5 is set by default, the set value a is set to the value of 0.5 by default. In the example shown in FIG. 3, since the preset switch 96 is pressed, in this case, the value set in the preset switch 96 in advance is set to the set value a. The value allocated to the preset switch 96 by default is 1.0. Similarly, if the preset switch 97 is pressed, the value set in the preset switch 97 in advance is set to the set value a. The value allocated to the preset switch 97 by default is 2.0.

In Step S100, if it is determined that the selection switch 92 is switched to the setter 91 side, subsequently, the CPU 101 reads a value VR of the setter 91 (Step S120). Then, processing for setting the set value a according to the read value VR is performed (Step S130). The value VR of the setter 91 is changed in a range of 0 to 100 by the position of a knob. A way to set the set value a with respect to the value VR of the setter 91 is arbitrary, and the set value a may be set by a function or a table may be prepared in advance and the set value a may be set referring to the table. As the function, for example, the following expression is established.

a=0.5+VR×0.015

Then, if VR varies from 0 to 100, the set value a is set within the same range as the range (0.5 to 2.0) of setting by the preset switches 95 to 97. Of course, if the following expression is established, when VR varies from 0 to 100, the set value a is set between 0.1 and 5.1 and can be set within a wider range than the range of setting by the preset switches 95 to 97.

a=0.1+VR×0.05

A way to set the set value a by the setter 91 may be determined assuming variation in the range of preference of each user or the like.

In Step S130, after the set value a is set according to the value VR of the setter 91 or in Step S110, after the set value a is set according to the state of the preset switch, a characteristic according to the set value a is displayed on the display unit 78 (Step S140), the process exits to “RTN”, and the interrupt routine ends. In regards to the display on the display unit 78, the relationship between the moving speed S of the ejection port 58 and the intensity of the liquid ejected from the ejection port 58 is displayed by the set value a. The relationship of FIG. 7 described above is displayed on the display unit 78.

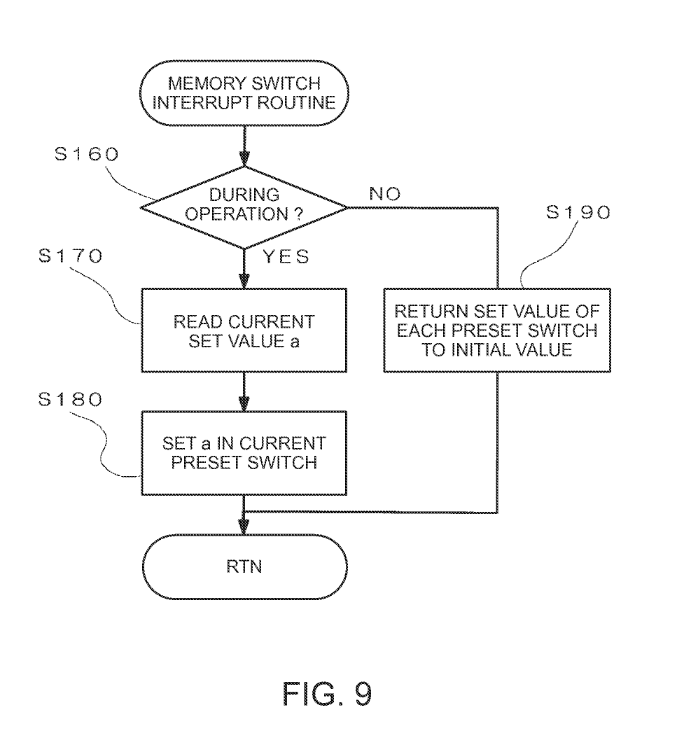

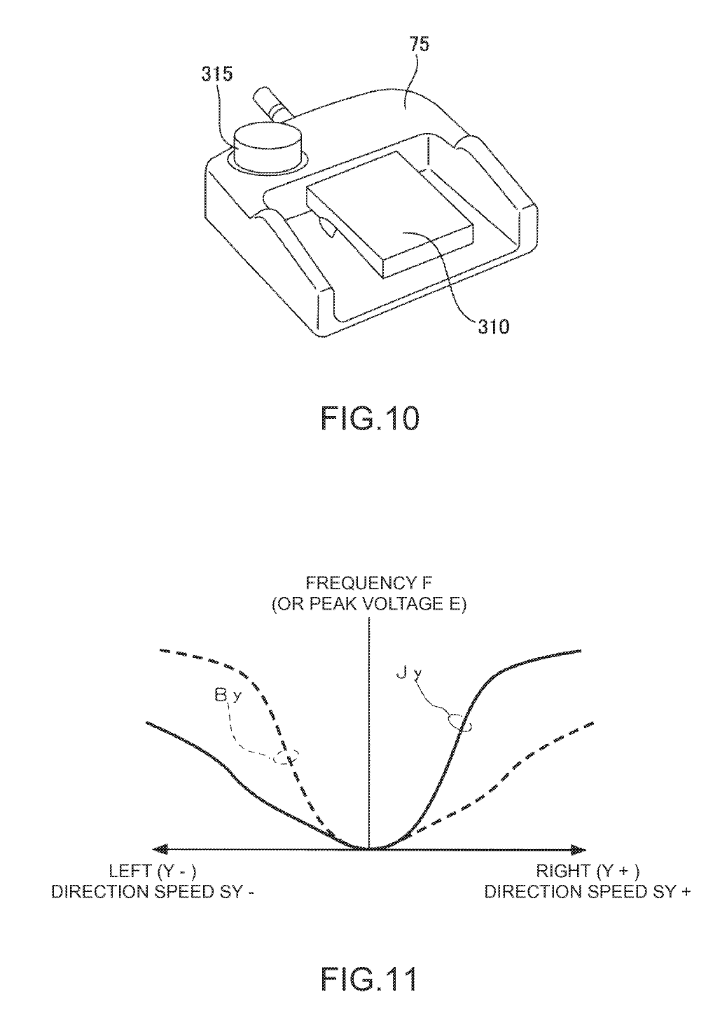

As described above, although the preset switches 95 to 97 are respectively set to the values of 0.1, 1.0, and 2.0 by default, in this embodiment, the preset values may be changed. This processing is shown in FIG. 9. FIG. 9 shows an interrupt routine which starts when the memory switch 94 provided in the operating unit 77 is operated. If this processing starts, first, the CPU 101 performs determination about whether or not the liquid ejecting apparatus 10 of this embodiment is during operation (Step S160). The term “during operation” means that the foot switch 75 is operated and the ejection of the liquid from the liquid ejecting mechanism 20 is performed. If it is determined to be during operation, next, processing for reading the current set value a is performed (Step S170). The set value a is a default value set in each of the preset switches 95 to 97 or a value set by the value VR of the setter 91.

Accordingly, the read set value a is set in the preset switches 95 to 97 currently being turned on (Step S180). With this processing, if the current set value a is the default value of each of the preset switches 95 to 97, the value is set in the preset switches 95 to 97 as it is, and if the current set value a is the value set by the setter 91, the value is set in the preset switches 95 to 97 currently being turned on. For example, when the selection switch 92 is switched to the setter side and the user operates the setter 91 to perform treatment with the set value a preferred by the user, if the memory switch 94 is operated, the set value a at this time is set in the preset switches 95 to 97 currently being turned on.

When the memory switch 94 is turned on and the interrupt routine of FIG. 9 is activated, if the liquid ejecting apparatus 10 is not during operation (Step S160: “NO”), processing for returning the set value of each of the preset switches 95 to 97 to an initial value is performed (Step S190). The set values are set to the values of 0.5, 1.0, and 2.0 already described. With this, it is possible to easily return the setting of each of the preset switches 95 to 97 to a default state. After the above-described processing, the process exits to “RTN”, and this interrupt routine ends.

### A5. Functional Effect of First Embodiment

According to the first embodiment described above, the following functional effects are obtained.

(1) Since the drive frequency F increases and decreases with a proportional relationship with respect to the speed S of the ejection port 58 of the liquid ejecting mechanism 20, even if the speed S varies, in any cases, variation of the energy per unit length is suppressed, and stable treatment is possible.

(2) The increase ΔF of the drive frequency F with respect to the increase ΔS of the speed S can be selected out of the three kinds set in the preset switches 95 to 97, and it is possible to flexibly cope with the preference of the user, a difference of a treatment target, or the like.

(3) The set value a can be freely set by the setter 91, and the relationship between the increase ΔS of the speed S and the increase ΔF of the drive frequency F by the set value a according to the preference of the user can be set.

(4) The set value a can be easily set in each of the preset switches 95 to 97 and can be simply called.

(5) The set value set in each of the preset switches 95 to 97 can be simply returned to the default value.

(6) As shown in FIG. 7, control can be performed such that the relationship between the increase ΔS of the speed S and the increase ΔF of the drive frequency F can be established in the treatment range SL to SH, and the pulsed ejection of the liquid is not performed outside the range.

### B. Second Embodiment

Next, a second embodiment will be described. A liquid ejecting apparatus 10 of the second embodiment has the same hardware configuration as in the first embodiment excluding the configuration of a foot switch 75. In the second embodiment, the foot switch 75 is provided with a selection switch 315 of a set value in addition to a pedal 310 configured to instruct ON and OFF of normal ejection. The selection switch 315 is a momentary on type switch, and each time the user presses the selection switch 315, an interrupt request is output to the control unit 70. If the interrupt request is received, the control unit 70 sets the set value a to one of the three values set in the preset switches 95 to 97 in regular order each time the selection switch 315 is operated. That is, each time the selection switch 315 is operated, the set value a is switched in the following order.

[1] the value set in the preset switch 95 (0.5 by default)
[2] the value set in the preset switch 96 (1.0 by default)
[3] the value set in the preset switch 97 (2.0 by default)

If the selection switch 315 is further operated, the set value a is switched in order from [1]. The switched set value a is displayed on the display unit 78 of the control unit 70 every time, and this is the same as the first embodiment.

According to the liquid ejecting apparatus 10 of the second embodiment configured as above, the set value a is switched by a simple operation to push down the selection switch 315 provided in the foot switch 75 familiar to the user, and the liquid ejecting mechanism 20 can be used in a desired mode. Accordingly, in addition to the same effects as the effects of the first embodiment, since the user does not necessarily operate the preset switches 95 to 97 of the control unit 70, there is a merit that user operation is simple.

### C. Modification Examples

The invention is not limited to the above-described embodiments, and can be of course carried out in various aspects.

Hereinafter, some of the modification examples are illustrated.

### C1. Modification Example 1

In the above-described embodiments, the drive frequency F is changed with respect to the speed S. The intensity of resection or incision by the liquid ejecting apparatus 10 may be controlled by the peak voltage of the drive signal, the rising time, the supply amount of the liquid to the liquid chamber, or the like, as well as the drive frequency. Accordingly, while the drive frequency F is kept constant, the relationship shown in FIG. 7 may be applied to the peak voltage E, the peak voltage E may be changed with respect to the speed S, and the relationship may be determined according to the set value a.

Alternatively, while the drive frequency F or the peak voltage E is kept constant, the relationship shown in FIG. 7 may be applied to the rising time of the drive signal, the rising time may be changed with respect to the speed S, and the relationship may be determined according to the set value a. The shorter the rising time, the stronger the resection or incision force.

Of course, instead of changing one of the parameters, such as the drive frequency, the peak voltage, the rising time of the drive signal, and the supply amount of the liquid to the liquid chamber, according to the speed S, a configuration in which a plurality of parameters are changed simultaneously or continuously in the range of the speed S may be introduced. At this time, the relationship of FIG. 7 may be applied to a plurality of parameters simultaneously and adjusted. For example, first, the drive frequency F may be changed according to the speed S, and then, after the drive frequency F exceeds the upper and lower limit values, another parameter, for example, the peak voltage may be changed. If a plurality of parameters are used, it is possible to further expand the variable range (dynamic range) of the intensity of resection or incision with respect to the speed S.

### C2. Modification Example 2

In the above-described embodiments, although the speed S is handled as the absolute value of the moving speed in the X and Y directions, since the acceleration sensor 69 can discriminate the direction, different set values may be used depending on the directions. For example, as shown in FIG. 11, the drive frequency F, the peak voltage E, or the like may be controlled while distinguishing between motion in the right direction (or upward direction) of the ejection port 58 of the liquid ejecting mechanism 20 and motion in the left direction (or downward direction). In the example shown in FIG. 11, the magnitude of the increase ΔF of the drive frequency F with respect to the increase ΔS of the speed S differs between motion in the right direction and motion in the left direction. In summary, the amount of change of the intensity of resection or incision with respect to the speed S differs between motion in the right direction and motion in the left direction.

The user is normally right-handed or left-handed, and a difference in characteristic according to handedness may be implemented. In FIG. 11, if a characteristic of a solid line Jy is preferred to a right-handed operator, it is assumed that a characteristic of a broken line By is preferred for a left-handed operator.

Alternatively, if a tissue of an internal organ as a treatment target has directivity, it is effective to change the characteristic accordingly. For example, when resection or incision of a muscle is performed, ease of resection or incision in a fibrous tissue direction of the muscle is different from ease of resection or incision in a direction intersecting the fibrous tissue. For example, if the characteristic differs in the X direction and the Y direction, resection or incision is facilitated.

### C3. Modification Example 3

As shown in FIG. 7, the setting of the set value a illustrated in the first embodiment is substantially linear in the use range SL to SH. The relationship between the speed S and the drive frequency (alternatively, the peak voltage or the like) is not necessarily linear, if a configuration in which a table is prepared, the relationship is stored in the table, and the table is looked up from the speed S is made, any relationships can be established.

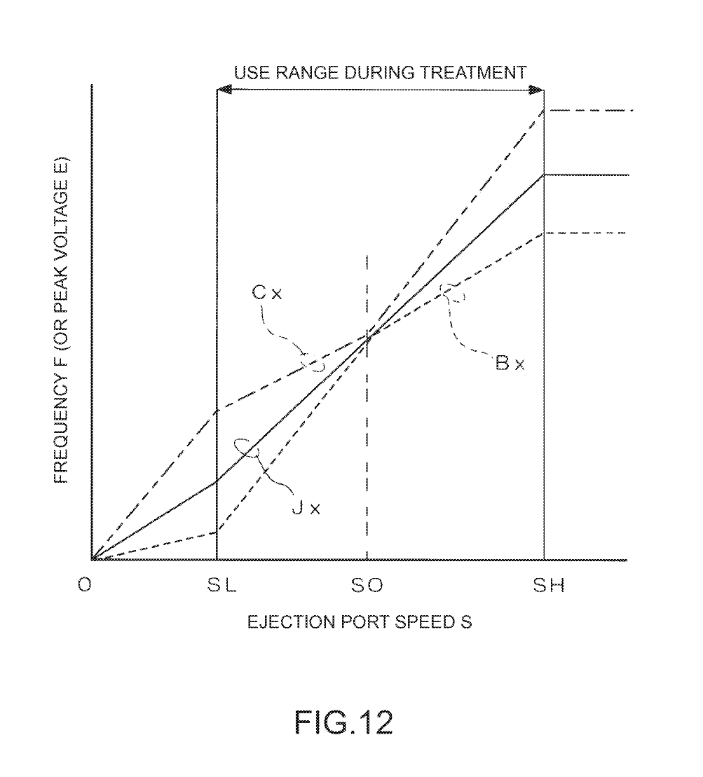

FIG. 12 shows an example of this relationship. In the first embodiment, the relationship between both is determined by a proportional coefficient, which is the set value a, and the drive frequency when the set value a is the value of 0.5, 1.0, and 2.0 is set to become the same value at the substantially center of the use range SL to SH of the ejection port speed S. For this reason, for example, when the set value a is the value of 2.0, and when the speed S is fast, the drive frequency F is greater than when the set value a is the value of 1.0. Meanwhile, when the speed S is slow, the drive frequency F is less than when the set value a is the value of 1.0. In contrast, in the setting example shown in FIG. 12, three setting examples are shown, and while the drive frequency F with respect to the speed S becomes equal at a substantially center speed SO as in the first embodiment (FIG. 7), the following points are different. That is, in the setting example shown in FIG. 12, if the relationship indicated by a broken line Bx is selected, the drive frequency F constantly falls below the relationship (the relationship of the set value a=1.0) indicated by a solid line Jx. If the relationship indicated by a two-dot-chain line Cx is selected, the drive frequency F constantly exceeds the relationship (the relationship of the set value a=1.0) indicated by the solid line Jx. With the relationship shown in FIG. 7, even if the relationships (solid line J, broken line B, and two-dot-chain line C) when the set value a=0.5, 1.0, and 2.0 intersect at the lower limit value SL of the speed range, the same relationship can be established.

If this setting is used, the intensity of resection or incision of the liquid ejected from the liquid ejecting mechanism 20 of the liquid ejecting apparatus 10 does not depend on speed, a setting Cx is constantly strongest, and a setting Bx is constantly weakest. For this reason, for example, when a treatment target is a comparatively soft tissue, such as a brain tissue, the setting Bx in which the intensity of resection or incision is lowest is selected, and when a treatment target is a comparatively touch tissue, such as a muscle, the setting Cx is selected. With this, it is possible to provide the same cutting quality with respect to the same speed S without depending on a treatment target.

### C4. Other Modification Examples

A parameter involved in the state of the liquid to be ejected is not limited to the ejection intensity, various parameters, such as the ejection amount of the liquid, the size of a liquid droplet to be intermittently ejected, and the duration of single ejection, may be used.

The intensity of ejection may be controlled by adjusting the representative volume of the liquid chamber 39 as well as the drive frequency or the peak voltage. The representative volume of the liquid chamber may be the volume of the liquid chamber 39 when no voltage is applied to the piezoelectric element 35 or may be the volume when a predetermined voltage is applied to the piezoelectric element 35. Alternatively, an average value may be used. The representative volume of the liquid chamber 39 can be easily changed by, for example, providing another piezoelectric element between the piezoelectric element 35 and the third case 33 and applying a voltage to the piezoelectric element to expand at a predetermined length. Of course, any configuration, for example, a configuration in which the volume of the liquid chamber 39 is variable may be introduced insofar as a configuration in which the volume of the liquid chamber 39 can be changed.

The drive waveform may be a combination of sinusoidal curves, and for example, may be increased or decreased in a stepwise manner.

The relationship between each of the peak voltage and the drive frequency and the speed of the ejection port may be defined in a curved manner or may be defined in a stepwise manner.

While the rising time is fixed, the drive frequency may be varied. That is, the time when the voltage of the drive signal falls down the peak and reaches zero may be changed, thereby varying the drive frequency. With this, when determining the drive frequency with respect to the moving speed, it is possible to exclude the influence of variation in the rising time, whereby the determination of the drive frequency is facilitated.

Although a case where the relationship between the amount of variation of the moving speed of the ejection port and the amount of change of the parameter involved in fluctuation of the state of the liquid can be set by the setting unit, the relationship between the moving speed of the ejection port and the parameter involved in fluctuation of the state of the liquid with respect to the moving speed of the ejection port may be stored. With this, even if ejection starts when the ejection port is moving, it is possible to perform desired liquid ejection.

The speed of the ejection port may be calculated by, for example, an acceleration sensor provided at the tip of the ejection port. In this case, it is considered that the calculation result is more correct.

Alternatively, the speed of the ejection port may be calculated using image processing. For example, a marker may be provided at the tip of the ejection port, and the movement of the marker may be captured by a camera, thereby calculating the speed of the ejection port.

When a robot operates the liquid ejecting apparatus, since the speed of the ejection port can be recognized by the robot, it is not necessary to calculate the speed of the ejection port, and the recognized value may be used. In addition to the moving speed of the affected part, the relative speed of the ejection port may be calculated. The measurement of the moving speed of the affected part may be attained by predicting or measuring motion by breathing or pulse.

The detection of the moving speed it not limited to the ejection port, and detection may be performed at a place which moves with the movement of the ejection port, or the moving speed of the liquid ejecting mechanism may be detected.

In this embodiment, although a case where the liquid ejecting mechanism 20 is a tool which is operated in the hand of the user has been described, the liquid ejecting mechanism 20 may be a tool which is operated into a living body as a liquid ejecting mechanism for use in an endoscope, such as a laparoscope.

The type of the acceleration sensor may be an electrostatic capacitance type or a heat detection type. The invention is not limited to the acceleration sensor, and a sensor which can detect the moving speed of the ejection port indirectly or directly may be used.

The liquid ejecting apparatus may be used other than a medical instrument.

For example, the liquid ejecting apparatus may be used in a cleaning apparatus which removes dirt by an ejected liquid.

The liquid ejecting apparatus may be used in a drawing apparatus which draws a line or the like by an ejected liquid.

A system for liquid ejection may be a system using laser light. An ejection system using laser light may be an ejection system which uses fluctuation in pressure by intermittently irradiating laser light onto a liquid and vaporizing the liquid.

It should be noted that the invention is not limited to the embodiments, the specific examples, and the modification examples described above, but can be implemented with a variety of configurations within the scope or the spirit of the invention. For example, the technical features in the embodiments, the specific examples, and the modification examples corresponding to the technical features in the aspects described in SUMMARY section can appropriately be replaced or combined in order to solve all or a part of the problems described above or in order to achieve all or a part of the advantages. Furthermore, the technical feature can appropriately be eliminated unless described in the specification as an essential component.

What is claimed is:

1. A liquid ejecting apparatus comprising:
a liquid ejecting mechanism which receives a drive signal as input and makes the pressure of a liquid in an internal liquid chamber fluctuate according to the drive signal to make the liquid be ejected from an ejection port,
wherein the drive signal is changed according to the moving speed of the liquid ejecting mechanism, and
the relationship between the amount of variation of the moving speed and the amount of change of the drive signal is able to be set.

2. The liquid ejecting apparatus according to claim 1, further comprising:
a user interface which receives an instruction of a set value; and
a setting unit which sets the relationship between the amount of variation of the moving speed and the amount of change of the drive signal according to the set value instructed by the user interface.

3. The liquid ejecting apparatus according to claim 1, further comprising:
a storage unit which stores a plurality of relationships between the amount of variation of the moving speed and the amount of change of the drive signal; and
a setting unit which sets the relationship between the amount of variation of the moving speed and the amount of change of the drive signal to one relationship selected from the storage unit.

4. The liquid ejecting apparatus according to claim 1,
wherein the drive signal includes at least one of the frequency of the drive signal and the voltage of the drive signal.

5. The liquid ejecting apparatus according to claim 1,
wherein, when the moving speed is a first speed, the drive signal is determined to be a first value, and when the moving speed is a second speed faster than the first speed, the drive signal is determined to be a second value at which power by the ejected liquid is higher than power of the first value.

6. The liquid ejecting apparatus according to claim 5,
wherein the setting unit sets the relationship between the increase of the second speed with respect to the first speed of the moving speed and the increase of the second value with respect to the first value of the drive signal.

7. The liquid ejecting apparatus according to claim 6,
wherein the setting unit sets one of a first mode, in which the increase ratio of the increase of the value and the increase of the moving speed is set to be less than 1, a second mode, in which the increase ratio is set to 1, and a third mode, in which the increase ratio is set to be greater than 1, according to information input by the user interface.

8. The liquid ejecting apparatus according to claim 1, further comprising:
a liquid supply unit which supplies the liquid to the liquid chamber,
wherein the supply flow rate of the liquid to the liquid chamber by the liquid supply unit is adjusted according to change of the drive signal.

9. A medical instrument comprising:
the liquid ejecting apparatus according to claim 1; and
a liquid supply unit which supplies the liquid to the liquid chamber,
wherein the liquid supply unit supplies a liquid for medical use to the liquid chamber.

10. A medical instrument comprising:
the liquid ejecting apparatus according to claim 2; and
a liquid supply unit which supplies the liquid to the liquid chamber,
wherein the liquid supply unit supplies a liquid for medical use to the liquid chamber.

11. A medical instrument comprising:
the liquid ejecting apparatus according to claim 3; and
a liquid supply unit which supplies the liquid to the liquid chamber,
wherein the liquid supply unit supplies a liquid for medical use to the liquid chamber.

12. A medical instrument comprising:
the liquid ejecting apparatus according to claim 4; and
a liquid supply unit which supplies the liquid to the liquid chamber,
wherein the liquid supply unit supplies a liquid for medical use to the liquid chamber.

13. A medical instrument comprising:
the liquid ejecting apparatus according to claim 5; and
a liquid supply unit which supplies the liquid to the liquid chamber,
wherein the liquid supply unit supplies a liquid for medical use to the liquid chamber.

14. The medical instrument according to claim 9,
wherein the liquid ejecting apparatus applies pulsation to the liquid to perform the ejection of the liquid.

15. The medical instrument according to claim 10,
wherein the liquid ejecting apparatus applies pulsation to the liquid to perform the ejection of the liquid.

16. The medical instrument according to claim 11,
wherein the liquid ejecting apparatus applies pulsation to the liquid to perform the ejection of the liquid.

17. The medical instrument according to claim 12,
wherein the liquid ejecting apparatus applies pulsation to the liquid to perform the ejection of the liquid.

18. The medical instrument according to claim 13,
wherein the liquid ejecting apparatus applies pulsation to the liquid to perform the ejection of the liquid.

19. A liquid ejecting apparatus comprising:
a liquid ejecting mechanism which receives a drive signal as input and makes the pressure of a liquid in an internal liquid chamber fluctuate according to the drive signal to make the liquid be ejected from an ejection port; and
a change unit which changes a parameter involved in fluctuation of the state of the liquid ejected from the ejection port according to the moving speed of the liquid ejecting mechanism,
wherein the relationship between the moving speed and the parameter corresponding to the moving speed is able to be set.

20. A liquid ejecting method which makes a liquid be supplied to a liquid chamber, makes a pressure in the liquid chamber fluctuate according to a drive signal, and makes the liquid of the liquid chamber be ejected from an ejection port at the tip of an ejection pipe by fluctuation of the pressure in the liquid chamber, the method comprising:
setting the relationship between the amount of variation of the moving speed of the ejection port and the amount of change of a parameter involved in fluctuation of the state of the liquid ejected from the ejection port; and
changing the parameter according to the moving speed using the set relationship.
